(19) United States
(12) Patent Application Publication
(10) Pub. No.: US 2011/0315140 A1
(43) Pub. Date: Dec. 29, 2011

(54) PORTABLE OXYGEN CONCENTRATOR

(75) Inventor: Aaron Jeffrey Shuman, Allentown, PA (US)

(73) Assignee: PRECISION MEDICAL, INC., Northampton, PA (US)

(21) Appl. No.: 12/826,144

(22) Filed: Jun. 29, 2010

(51) Int. Cl. A61M 16/10; B01D 53/047

(52) U.S. Cl. 128/204.23; 128/205.11; 128/204.21; 128/205.24; 96/113; 96/114; 96/121

(57) ABSTRACT

A vacuum pressure swing absorption system provides portable oxygen to medical patients. A pair of sieve beds are alternately pressurized with air and evacuated by vacuum by coordinated operation of a selector valve and a vent valve. Product gas flows from both sieve beds through respective check valves to a product tank. An equalization valve is operated in coordination with the selector valve and the vent valve to open a flow path between the sieve beds and allow pressure to equalize therebetween. Operation of the vent valve is time to occur after pressure in the sieve beds has equalized. Components of the concentrator are tuned so that the equalization pressure occurs at about zero pounds per square inch (psi). This reduces energy loss due to free expansion of gasses and reduces noise created by the venting of gasses to the ambient atmosphere.

### FIELD OF THE INVENTION

The present disclosure relates to vacuum pressure swing absorption (VPSA) systems that separate portions of a gas mixture, and in particular to an improved portable oxygen concentrator to produce oxygen-enriched gas for medical patients.

### BACKGROUND OF THE INVENTION

The use of portable systems for delivering substantially pure oxygen to ambulatory patients has seen substantial growth in recent years. Early examples of such systems used small highly pressurized oxygen cylinders which were wheeled or carried around by patients. More advanced systems make use of liquid oxygen which travels through suitable warming coils before being inhaled by the patient.

Other portable oxygen systems convert air to substantially pure oxygen by fractionating the air. More specifically, a compressor draws air in from the ambient environment and pushes it through a pair of molecular sieve beds. These sieve beds contain a material such as zeolite, which removes nitrogen from the air and causes substantially pure oxygen to exit from the sieve beds. These systems, known as oxygen concentrators, generally make use of molecular sieve beds and use a four-part cycle, known as the pressure swing adsorption cycle (PSA) or vacuum pressure swing absorption cycle (VPSA).

One of the drawbacks of current portable oxygen concentrators is maintaining a consistent bolus volume delivered in response to patient inhalation. Current systems generally use elaborate or multiple lookup tables which will open an oxygen delivery valve for a certain period of time depending on which setting has been selected on the unit. This complicates the design and programming of the systems used to deliver oxygen pulses in response to patient inhalation. Other attempts to provide a consistent bolus volume have had mixed results, disadvantages or drawbacks.

Another disadvantage common to presently known oxygen concentrators which utilize the PSA cycle is that such apparatus emit an audible venting sound which can become annoying to the users and bystanders. Portable concentrators generally use pressure to charge the sieve beds, but then simply vent the sieve beds to atmosphere to purge them as required by the fractionating process. This purging to atmosphere creates the audible venting sound. These apparatus also suffer from high energy consumption due in part to energy losses inherent in free expansion of gasses upon opening the vent valve separating disparate pressure environments.

Those systems that have attempted to address the foregoing drawbacks have been complex, expensive, or suffer from other drawbacks and disadvantages. Portable concentrators of the current art also often, disadvantageously, make use of user-unfriendly controls, featuring an array of buttons.

### SUMMARY OF THE INVENTION

Illustrative embodiments of the present invention provide a lightweight, inexpensive portable oxygen concentrator which delivers a consistent bolus volume in response to patient inhalation.

In an illustrative embodiment, a portable oxygen concentrator includes a pair of sieve beds which are alternately pressurized with air and evacuated by vacuum by coordinated operation of a selector valve and a vent valve. Product gas flows from both sieve beds through respective check valves to a product tank. An equalization valve is operated in coordination with the selector valve and the vent valve to open a flow path between the sieve beds and allow pressure to equalize there between. Operation of the vent valve is timed to occur at the same time as the equalization valve. Components of the concentrator are tuned so that the equalization pressure occurs at about zero pounds per square inch (psi).

The illustrative embodiments make use of a conserver reservoir in fluid communication with the product tank via an orifice. In an embodiment of the invention, a supply valve on the sieve bed portion of the inventive concentrator is controlled as function of pressure in the product tank.

Another illustrative embodiment of the invention provides a method for improved delivery of oxygen to a medical patient. The method includes pressurizing a first sieve bed with air while substantially simultaneously evacuating a second sieve bed by applying a vacuum device to the second sieve bed for a first cycle portion. During the first cycle portion, gas from the first sieve bed that exceeds a pressure within a product tank in fluid communication with the first sieve bed is allowed to flow from the first sieve bed to the product tank. In response to completion of the first cycle portion, fluid communication between the vacuum device and the second sieve bed is disconnected, and substantially simultaneously a fluid flow path between the first sieve bed and the second sieve bed is opened to perform a second cycle portion until the first sieve bed and the second sieve bed are at about equal pressure. In response to completion of the second cycle portion, the second sieve bed is pressurized with air while the first sieve bed is substantially simultaneously evacuated by applying the vacuum device to the first sieve bed for a third cycle portion. During the third cycle portion, gas from the second sieve bed that exceeds a pressure within a product tank in fluid communication with the second sieve bed is allowed to flow from the second sieve bed to the product tank. In response to completion of the third cycle portion, fluid communication between the vacuum device and the first sieve bed is disconnected, and substantially simultaneously a fluid flow path between the first sieve bed and the second sieve bed is opened to perform a fourth cycle portion until the first sieve bed and the second sieve bed are at about equal pressure.

Another illustrative embodiment of the invention provides a vacuum pressure swing absorption (VPSA) type oxygen concentrator. The embodiment includes a first VPSA sieve bed connected to a product tank for supplying concentrated oxygen to the product tank. The first VPSA sieve bed includes an input port configured to be closed or to receive either pressurized air or vacuum in response switching of at least one valve. A second VPSA sieve bed is connected to the product tank for supplying concentrated oxygen to the product tank. The second VPSA sieve bed includes an input port configured to be closed or to receive either pressurized air or vacuum pressure in response to switching of the at least one valve.

In the illustrative embodiment, the first VPSA sieve bed is in switchable fluid communication with the second VPSA sieve bed via the at least one valve. Control circuitry in communication with at least one valve is configured to switch the at least one valve to open the switchable fluid communication between the first VPSA sieve bed and the second VPSA sieve bed and to allow pressure to equalize between the first VPSA sieve bed and the second VPSA sieve bed. The control circuitry is configured to switch the at least one valve between pressuring the first VPSA sieve bed and pressurizing the second VPSA sieve bed substantially simultaneously with pressure in the first VPSA sieve bed equaling pressure in the second VPSA sieve bed.

### BRIEF DESCRIPTION OF THE DRAWINGS

The foregoing and other features and advantages of the present invention will be more fully understood from the following detailed description of illustrative embodiments, taken in conjunction with the accompanying drawings in which:

FIGS. 1A-1D show a system schematic diagram of a portable oxygen concentrator system during operation of a four step cycle according to an illustrative embodiment of the present invention;

FIG. 2 is a graph of first sieve bed pressure and second sieve bed pressure versus time during a four step cycle of operation according to an illustrative embodiment of the invention;

FIG. 3 is a valve timing diagram for switching valve states during the four step cycle of operation according to an illustrative embodiment of the invention;

FIG. 4 is a graph of product tank pressure versus time showing trigger points upon which the valve timing is based in an illustrative embodiment of the invention;

FIG. 5 is an example of a look up table including valve actuation times for controlling a 2-way conserver valve according to an illustrative embodiment of the invention;

FIGS. 6A and 6B show graphs of product tank pressure versus time in systems having no conserver reservoir and orifice configuration with systems having a conserver reservoir and orifice in accordance with illustrative embodiments of the invention;

FIG. 7 is an exploded view of a portable oxygen concentrator product tank assembly according to an illustrative embodiment of the invention; and

FIG. 8 is an exploded view of a portable oxygen concentrator apparatus according to an illustrative embodiment of the invention.

### DETAILED DESCRIPTION OF THE DRAWINGS

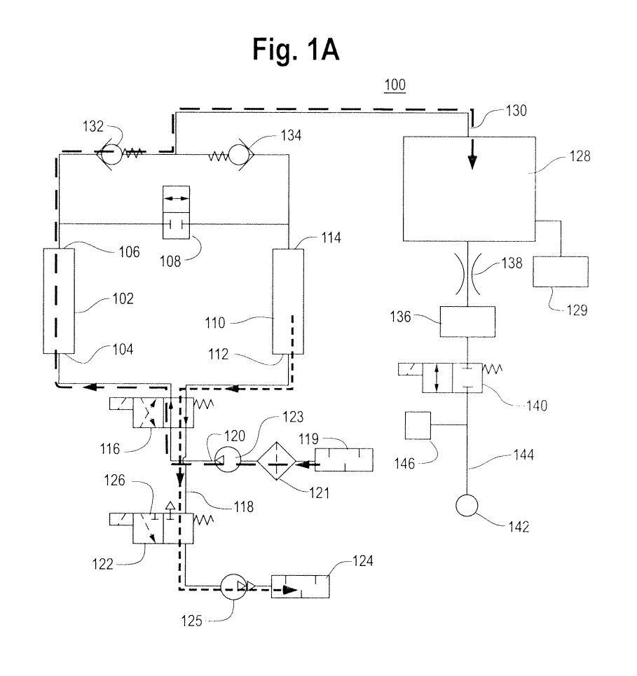

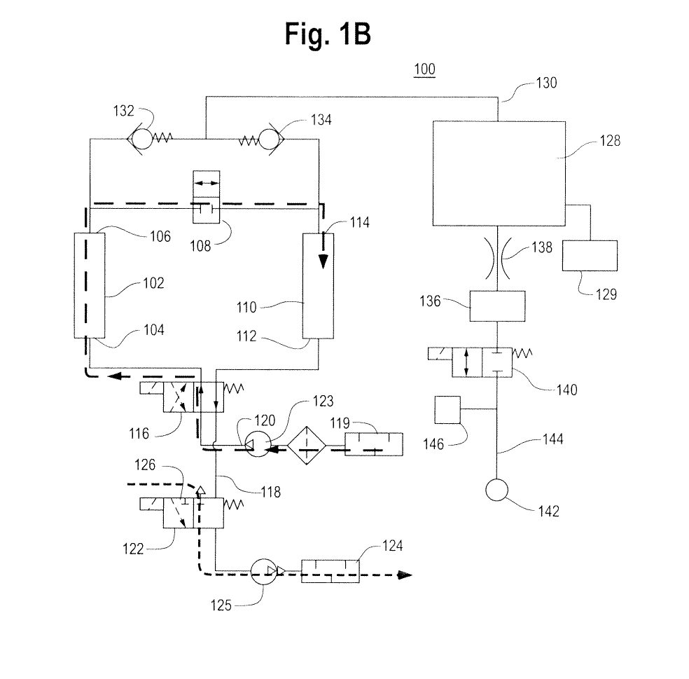

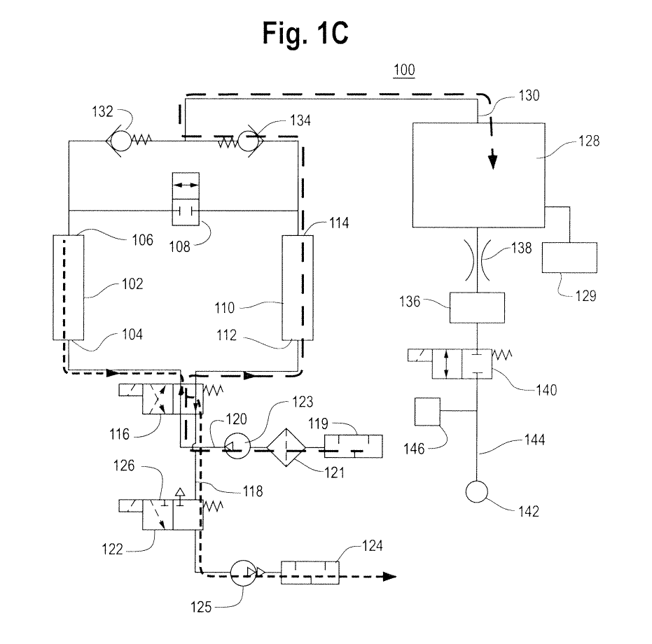

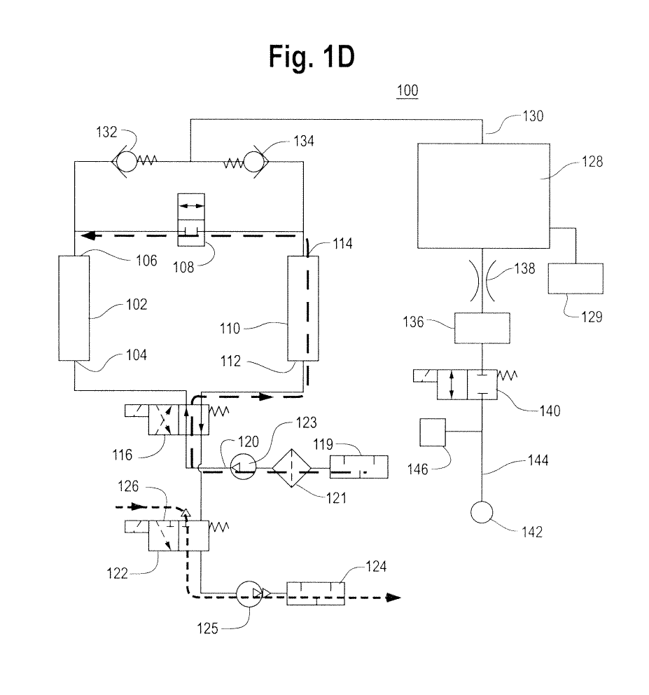

Turning to the drawings, FIGS. 1A-1D show a system schematic diagram of a portable oxygen concentrator system during a four step cycle according to an illustrative embodiment of the present invention.

The system 100 includes a first sieve bed 102 including a first pressure vessel having a first inlet port 104 and a first outlet port 106. The first outlet port 106 is connected to a 2-way equalization valve 108.

A second sieve bed includes a second pressure vessel having a second inlet port 112 and a second outlet port 114. The second outlet port 114 is also connected to the 2-way equalization valve 108. The 2-way equalization valve 108 has a first state providing a flow path between the first outlet port 106 and the second outlet port 114, and has a second state closing the flow path between the first outlet port 106 and the second outlet port 114.

A 4-way selector valve 116 is connected to the first inlet port 104, the second inlet port 112, a vacuum path 118 and a compressed air path 120. The 4-way selector valve has a first state providing a flow path between the compressed air path 120 and the first inlet port 104 and providing a flow path between the vacuum path 118 and the second inlet port 112. The 4-way selector valve has a second state providing a flow path between the compressed air path 120 and the second inlet port 112 and providing a flow path between the vacuum path 118 and the first inlet port 104.

Atmospheric air is drawn into a compressed air path 120 by a compressor 123. In an illustrative embodiment the compressor 123 draws air through at least one muffler 119 and filter 121. A 3-way vent valve 122 is connected in the vacuum path 118 between a vacuum source 125 and the 4 way selector valve 116. Exhaust from the vacuum source 125 may be passed through a muffler 124 to atmosphere. The 3-way vent valve 122 also has a port 126 open to ambient atmospheric pressure. The 3-way vent valve 122 has a first state providing a flow path between the vacuum path 118 and the vacuum source 125 and has a second state providing a flow path between the vacuum source 125 and the port 126 open to atmospheric pressure while closing the vacuum path 118.

A product tank 128 has at least one product tank input port 130 connected to the first outlet port 106 through a first check valve 132 and connected to the second outlet port 114 through a second check valve 134. The first check valve is arranged to allow flow from the first sieve bed 102 to the product tank 128 when pressure in the first sieve bed 102 exceeds pressure in the product tank 128. The second check valve 134 is arranged to allow flow from the second sieve bed 110 to the product tank 128 when pressure in the second sieve bed 110 exceeds pressure in the product tank 128.

The illustrative embodiment also includes a conserver reservoir 136 connected to the product tank 128 via an orifice 138. A 2-way conserver valve 140 is connected between the conserver reservoir 136 and a patient interface 142. The 2-way conserver valve 140 has a first state providing a flow path 144 between the conserver reservoir 136 and the patient interface 142, and has a second state closing the flow path 144 between the conserver reservoir 136 and the patient interface 142.

In the illustrative embodiment, control circuitry is arranged for repeatedly switching from a first cycle portion to a second cycle portion to a third cycle portion to a fourth cycle portion. In the first cycle portion, the circuitry sets the 2-way equalization valve 108 to its first state, the 4-way selector valve 116 to its first state and the 3-way vent valve 122 to its first state. In the second cycle portion, the circuitry sets the 2-way equalization valve 108 to its second state, the 4-way selector valve 116 to its first state and the 3-way vent valve 122 to its second state. In the third cycle portion, the circuitry sets the 2-way equalization valve 108 to its first state, the 4-way selector valve 116 to its second state and the 3-way vent valve 122 to its first state. In the fourth cycle portion, the circuitry sets the 2-way equalization valve 108 to its second state, the 4-way selector valve 116 to its second state and the 3-way vent valve 122 to its second state.

In one illustrative embodiment, the circuitry is configured for completing at least one of the first cycle portion, the second cycle portion, the third cycle portion and the fourth cycle portion after a respective fixed time period. In another illustrative embodiment, the circuitry is configured for completing at least one the first cycle portion, the second cycle portion, the third cycle portion and the fourth cycle portion after pressure in the product tank 128 reaches a predetermined threshold.

The states and functions of the various components of the illustrative embodiment during the first cycle portion, the second cycle portion, the third cycle portion and the fourth cycle portion are now further described with reference to FIGS. 1A-1D.

The first cycle portion is described with reference to FIG. 1A wherein the 4-way selector valve 116 is in its first state so first sieve bed 102 is pressurized with air from compressor 123. The 3-way vent valve 122 is also in its first state so the second sieve bed 110 is substantially simultaneously evacuated by the vacuum device 125. High purity oxygen from the first sieve bed 102 that exceeds a pressure within the product tank 128 in fluid communication with the first sieve bed 102 is allowed to flow from the first sieve bed 102 to the product tank 128 via check valve 132. In the first cycle portion, equalization valve 108 is in its first state so gas can not flow between the first sieve bed 102 and the second sieve bed 110. In the illustrative embodiment, the first cycle portion is complete, as indicated by expiration of a predetermined time or by measurement of a threshold pressure in the product tank 128.

The second cycle portion is described with reference to FIG. 1B. The first sieve bed 102 reaches its maximum pressure and the second sieve bed 110 reaches its maximum vacuum. The compressor 123 continues to feed air into the first sieve bed 102 through the 4-way selector valve 116. The 3-way vent valve 122 is switched to its second state. This opens the vacuum device 125 to the ambient atmosphere and closes vacuum path 118 to the second sieve bed 110. The 2-way equalization valve 108 is also switched to its second state. This allows oxygen enriched air to flow from the pressurized first sieve bed 102 to the evacuated second sieve bed 110 until the two sieve beds are at substantially equal pressure. In the illustrative embodiment, the various system components, including the compressor and the vacuum device, are selected and configured so that the equalization pressure occurs at about zero psi.

This equalization serves to transfer what would be wasted concentrated oxygen from one sieve bed to another and also prepares the sieve beds for the next step by bringing them both to near zero pressure.

The third cycle portion is described with reference to FIG. 1C. In response to completion of the second cycle portion, i.e., when the equalization pressure of about zero psi is reached in each of the sieve beds 102, 110, the 3-way vent valve 122 and the 2-way equalization valve 108 are both switched hack to their first state. The 4-way selector valve 116 is switched from its first state to its second state. This pressurizes the second sieve bed 110 with air from compressor 123 while substantially simultaneously evacuating the first sieve bed 102 by applying the vacuum device 123 to the first sieve bed 102. During the third cycle portion, enriched gas from the second sieve bed 110 that exceeds a pressure within a product tank 128 in fluid communication with the second sieve bed 110 is allowed to flow from the second sieve bed 110 to the product tank 128 via check valve 134. In the illustrative embodiment, the third cycle portion is complete, as indicated by expiration of a predetermined time or by measurement of a threshold pressure in the product tank 128.

The fourth cycle portion is described with reference to FIG. 1D. The second sieve bed 110 reaches its maximum pressure and the first sieve bed 102 reaches its maximum vacuum. The compressor 123 continues to feed air into the second sieve bed 102 through the 4-way selector valve 116. The 3-way vent valve 122 is switched to its second state. This opens the vacuum device 125 to the ambient atmosphere and closes vacuum path 118 to the first sieve bed 102. The 2-way equalization valve 108 is also switched to its second state. This allows oxygen enriched air to flow from the pressurized second sieve bed 110 to the evacuated first sieve bed 102 until the two sieve beds are at equal pressure.

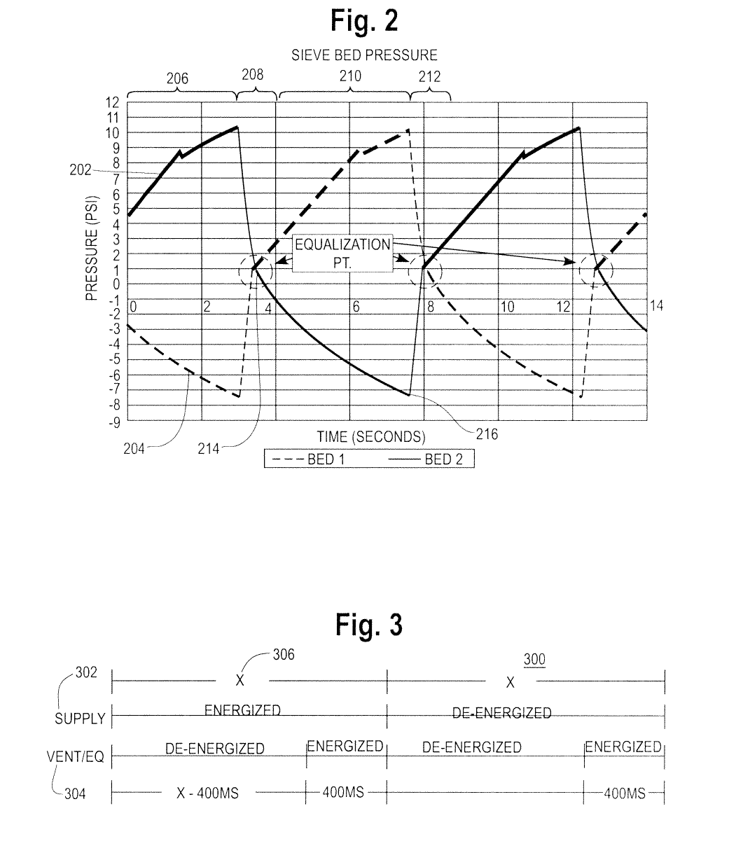

FIG. 2 is a graph 200 of first sieve bed pressure 202 and second sieve bed pressure 204 versus time during the first cycle portion 206, the second cycle portion 208, the third cycle portion 210 and the fourth cycle portions 212 of the illustrative embodiment. The pressure curves intersect at equalization points 214 at the end of the second cycle portion and equalization point 216 at the end of the fourth cycle portion 216. Although other configurations are possible, in this illustrative embodiment, the equalization points occur at about zero psi.

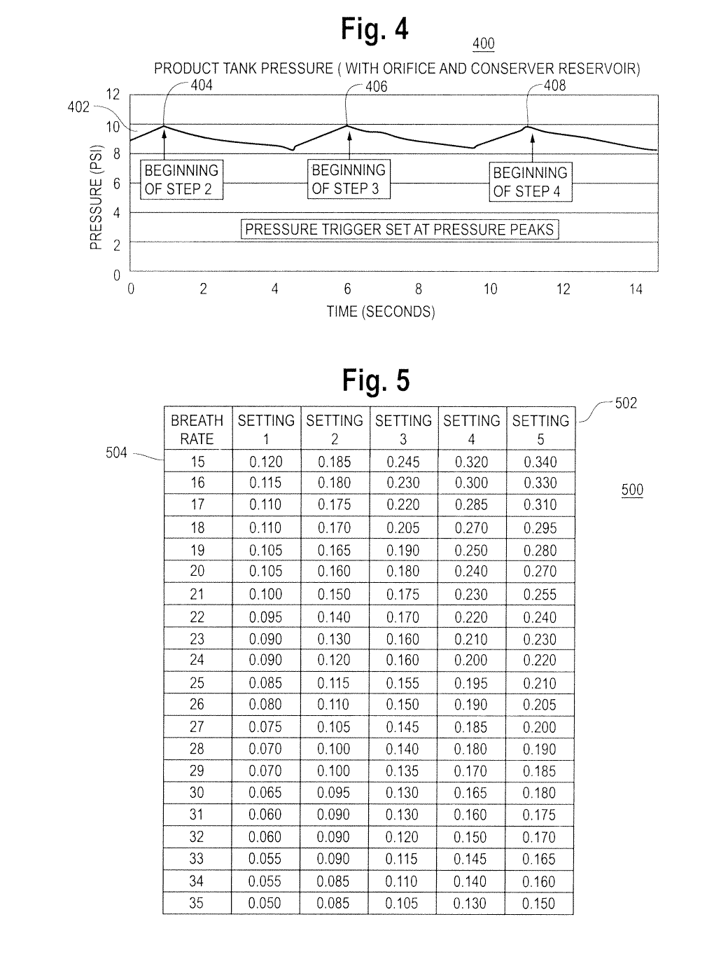

FIG. 3 is a cycle timing diagram 300 for switching states of the 4-way selector valve 116 as shown by line 302, and states of the 3-way vent valve 122 and 2-way equalization valve 108 as shown by line 304. In one embodiment the valves may be switched based on a fixed time in which time “X” 306 does not change. In another embodiment, the valves may be switched based on a pressure trigger, such that time “X” is allowed to vary. FIG. 4 is a graph 400 of product tank pressure 402 versus time showing trigger points 404, 406, 408 upon which the valve timing is based. For example, peaks in product tank pressure 402 as measured by the product tank pressure transducer 129 may be used to trigger the beginning of the first cycle portion, second cycle portion, third cycle portion, for example.

Referring again to FIGS. 1A-1D, in the illustrative embodiment, the conserver reservoir 136, product tank 128, orifice 138, and 2-way conserver valve 140, are configured to provide a consistent bolus volume over time. The orifice 138 reduces pressure variation in the conserver reservoir 136. At least one patient interface pressure transducer 146 is connected to the patient interface 142 and arranged to measure pressure in the patient interface 142. Patient interface pressure transducer 146 is used to sense inhalation from the user and control triggering of the conserver valve 140. The open time of the conserver valve 140 controls the bolus volume. In the illustrative embodiment, the conserver valve 140 is controlled using a single lookup table of valve actuation times. An example of a lookup table including times for controlling the conserver valve 140 is provided in FIG. 5. The time values in FIG. 5 may be chosen to provide a volume of oxygen per minute (sometimes referred to as “minute volume”) which achieves appropriate patient oxygen saturation levels regardless of changes in the patient's breath rate. Valve actuation times are determined by cross referencing a device setting 502 such as setting 1-setting 5 selected by the user with a user's breath rate 504. The breath rate may be calculated by averaging the breath rates during a number of trailing breaths. In the illustrative embodiment, setting 1 provides the lowest oxygen output (cc/min) and setting 5 provides the greatest oxygen output (cc/min). Compressor speed and trigger pressure are increased as the setting increases. This enables the system to use the least amount of energy for the required oxygen output. Other embodiments using different lookup tables, multiple lookup tables, and different settings are likewise within the scope of this disclosure.

Illustrative embodiments of the present invention ensure consistency and repeatability of the bolus (pulse) delivered by the conserver portion of the system. The bolus is created by opening the conserver valve 140 for the prescribed period of time, for example as prescribed in the lookup table of shown in FIG. 5. For a given valve actuation time, any variation of pressure feeding the valve results in a corresponding variation in the volume of the bolus. This can result in significant pressure fluctuations which cause the bolus volume to vary from breath to breath. The orifice 138 and conserver reservoir 136 reduce such variation because they provide the conserver valve 140 with a more consistent pressure.

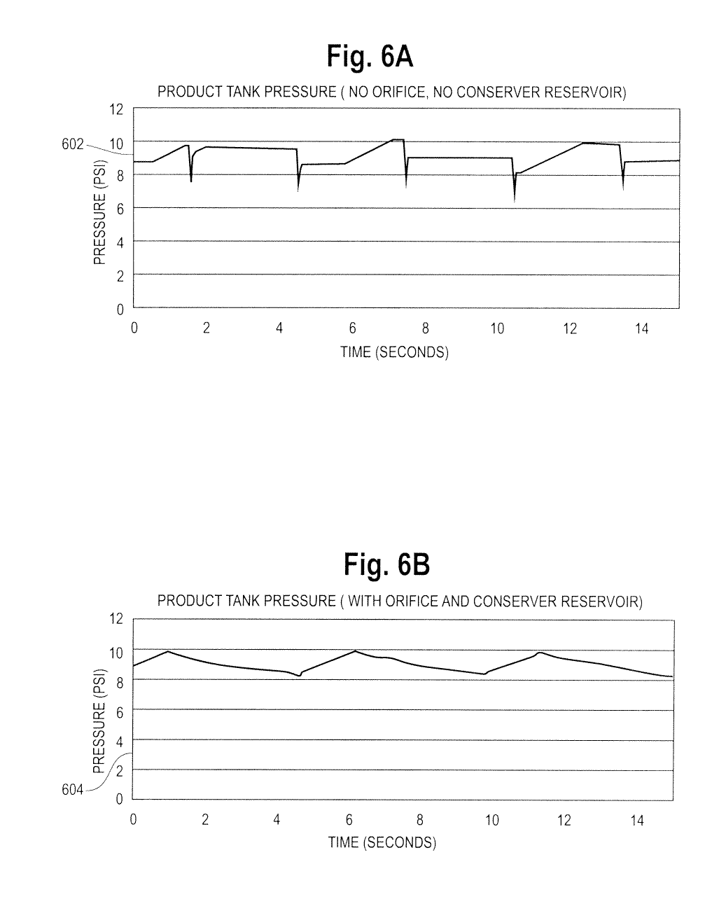

Referring to FIGS. 6A-6B, the use of an orifice and conserver reservoir in accordance with the illustrative embodiment of the invention transforms the pressure in the product tank from an erratic unpredictable curve 602 (FIG. 6A) to a gentle and repeatable saw tooth waveform 604 (FIG. 6B). In one possible implementation, at least one product tank pressure transducer 129 is arranged to measure pressure in the product tank 128. The product tank pressure transducer 129 may be operatively connected such as by programmable logic circuitry to control switching of the 4-way selector valve 116, the 3-way vent valve 122, and the equalization valve 108.

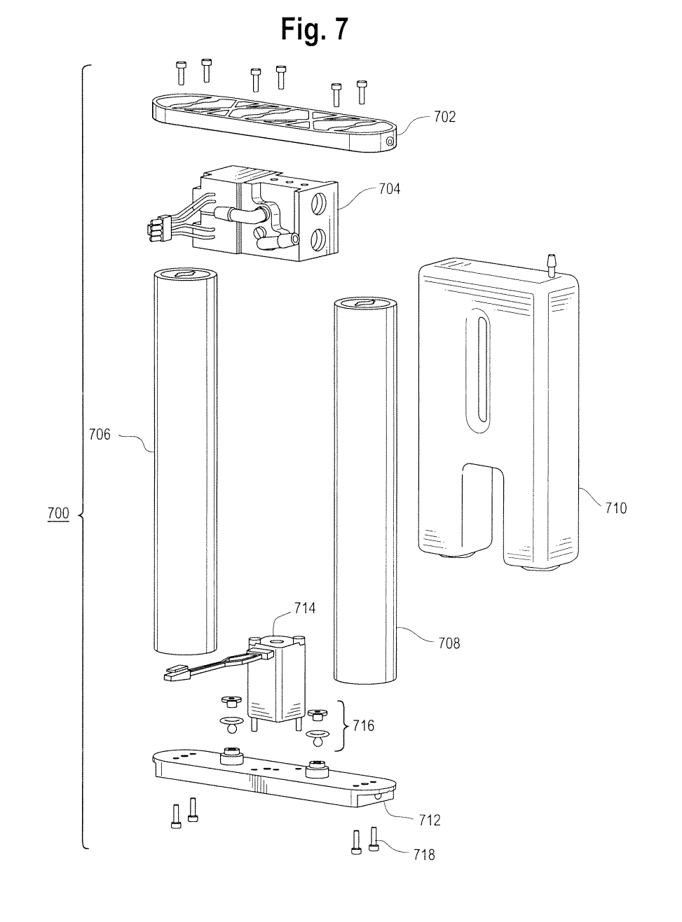

In the illustrative embodiment, the system described herein is constructed as a lightweight portable apparatus which is designed for simplicity and manufacturability. Referring to FIG. 7, and FIGS. 1A-1D, the apparatus includes a product tank assembly 700 a supply manifold 702 which is fastened to a main valve assembly 704. The main valve assembly 704 includes the 4-way selector valve 116 and the 3-way vent valve 122 in a single unit. The supply manifold 702 includes flow channels for both the compressed air path 120 and vacuum path 118 and also includes flow paths between the 4-way selector valve main valve assembly 704 and the sieve beds 706, 708.

The product tank 710 is disposed between the sieve beds 706, 708 and is in fluid communication with the sieve beds 706, 708 via the oxygen manifold 712. The oxygen manifold 712 includes flow paths for oxygen from the sieve beds 706, 708 to the product tank 710 and between the sieve beds 706, 708 via the equalization valve 714 which is mounted onto the oxygen manifold 712. The product tank 710 is shaped to straddle the equalization valve 714. The check valves between sieve beds 706 and 708 are integrally constructed in the oxygen manifold 712. The valve assemblies 704, 714, sieve beds 706, 708 and product tank 710 are fitted neatly together between the supply manifold 702 and oxygen manifold 712 and are secured in place with suitable fasteners 718.

In the illustrative embodiment, the product tank 710 is blow-molded and carefully constructed to fill the available space between the sieve beds 706, 708. Forming of the product tank 710 required an ultrasonic welding operation to form a sufficiently tight fit with the input orifices in the oxygen manifold 712.

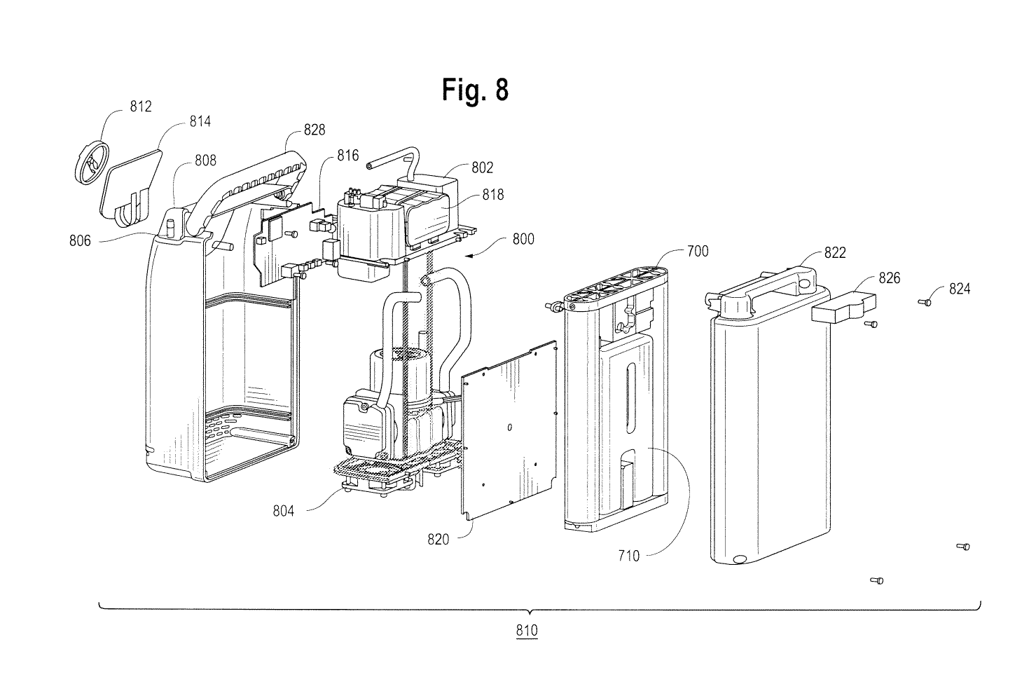

Referring to FIG. 8, the product tank assembly 700 is assembled to a separate assembly 802 which holds a conserver reservoir assembly 802 and a compressor assembly 804. The compressor assembly 804 includes the compressor and vacuum device that were described herein with reference to FIGS. 1A-1D. The conserver reservoir assembly 802 includes the conserver reservoir which is placed in fluid communication with the product tank 710 via an orifice (138 FIGS. 1A-1D) when the separate assembly 800 is assembled to the product tank assembly 700. A 2-way conserver valve (140 FIGS. 1A-1D) which controls flow of product gas between the conserver reservoir and a patient interface is also included in the conserver reservoir assembly 802. A battery 802, such as a lithium ion battery, is mounted in conserver reservoir assembly 802. The battery provides power for the compressor, vacuum device and control electronics of the apparatus.

The patient interface includes an outlet fitting 806 in a front case portion 808 of the apparatus 810. A control knob 812 mounted to the front case portion 808 allows the user to select from 1 of 5 settings. In an illustrative embodiment, a control membrane 814 is also mounted to the front case portion 808. The control membrane 814 may include a rotary switch actuated by the control knob 812, and/or it may provide separate switching functionality for additional control features. A printed circuit board 816 mounted inside the front case portion 808 provides connectivity between the control membrane and/or the rotary switch and control circuitry which control the valve timing, and compressor speeds as described above for a given setting and breath rate. The control circuitry may include programmable logic circuitry such as a processor and memory which may be mounted on the printed circuit board 816. A rigid plate 820 encloses the compressor and vacuum device within the front case portion 808 to form a compressor compartment therein. The product tank assembly 700 is mounted behind the compressor compartment.

A rear case portion 822 is secured by an appropriate number of fasteners 824 to the front case portion 818 to completely enclose the apparatus. A filter 826 is easily accessible through the rear case portion 822 for installation or replacement of the filter 826 without disassembly of the front and rear case portions 808, 822. A handle 828 may also be attached to the front or rear case portions 808, 822.

Operation of the illustrative embodiments is apparent from the foregoing description. Illustratively, a patient's oxygen tube from an oxygen mask, breathing tube, or the like is connected to the outlet fitting 806. The portable oxygen concentrator is turned on by turning the knob 812 to one of five settings, for example. Control circuitry on the printed circuit board, including memory for storing program instructions and at least one processor, controls the speed of the compressor and/or vacuum circuitry in response to the knob setting and in accordance with the stored program instructions. In accordance with the stored program instructions, sensor input and/or lookup tables, the control circuitry also automatically energizes and dc-energizes the 4-way selector valve 116, the 3-way vent valve 122, the 2-way equalization valve 108, and the conserver valve 140 (FIGS. 1A-1D) to switch the valves between their respective first and second states at the appropriate times. A medical patient using the device may then breathe through their mask, breathing tube, or the like. The portable oxygen concentrator does not require connections to external oxygen sources, vacuum sources or electrical power so it may be carried during use by handle 828.

Illustrative embodiments of the invention reduce energy loss due to free expansion of gasses and reduce noise created by the venting of gasses to the ambient atmosphere. The “tuned” equalization point near zero psi allows the use of a relatively simple valve arrangement while still allowing efficient use of the vacuum pump which releases waste air to the atmosphere.

The sieve material itself works best when it cycles through a pressure range centered around zero psi, leading to more efficient use of the sieve material. Power consumption and energy loss of the device is reduced if the moment of changeover from pressurizing to purging of the sieve beds occurs when the equalization point is close to zero.

Sieve bed degradation over time, changes in altitude or variations in compressor performance may be compensated for by adjusting the cycle in response to monitoring pressure in the product tank.

Illustrative embodiments of the invention use a relatively small number of parts and employ a simple pressure vacuum cycle which saves cost, weight and size. The embodiments do not require complicated programming or use of multiple lookup tables. Control inputs are simplified for users by using a selector knob with detent settings to select from a simple set of control levels.

Although various embodiments of the invention are described herein with reference to an end user being a medical patient, it should be understood by persons having ordinary skill in the art that the term “medical patient” refers to any end user of the disclosed systems, methods and apparatus. While the invention has been described with reference to illustrative embodiments, it will be understood by those skilled in the art that various other changes, omissions and/or additions may be made and substantial equivalents may be substituted for elements thereof without departing from the spirit and scope of the invention. In addition, many modifications may be made to adapt a particular situation or material to the teachings of the invention without departing from the scope thereof. Therefore, it is intended that the invention not be limited to the particular embodiment disclosed for carrying out this invention, but that the invention will include all embodiments falling within the scope of the appended claims. Moreover, unless specifically stated any use of the terms first, second, etc. do not denote any order or importance, but rather the terms first, second, etc. are used to distinguish one element from another.

What is claimed is:

1. A method for improved delivery of oxygen to a medical patient comprising:
providing a vacuum pressure swing absorption (VPSA) oxygen concentrator apparatus including a first sieve bed and a second sieve bed; and
alternatively switching between pressurizing the first sieve bed and the second sieve bed when an equalization point of about zero pounds per square inch (psi) in said first sieve bed and said second sieve bed is reached in a VPSA cycle of said oxygen concentrator apparatus.

2. The method of claim 1, comprising:
pressurizing the first sieve bed with air while substantially simultaneously evacuating the second sieve bed by applying a vacuum device to the second sieve bed for a first cycle portion;
during said first cycle portion, allowing gas from said first sieve bed that exceeds a pressure within a product tank in fluid communication with the first sieve bed to flow from the first sieve bed to the product tank;
in response to completion of the first cycle portion, disconnecting fluid communication between the vacuum device and the second sieve bed, and substantially simultaneously opening a fluid flow path between the first sieve bed and the second sieve bed to perform a second cycle portion until the first sieve bed and the second sieve bed are at about equal pressure;
in response to completion of the second cycle portion, pressurizing the second sieve bed with air while substantially simultaneously evacuating the first sieve bed by applying the vacuum device to the first sieve bed for a third cycle portion;
during the third cycle portion, allowing gas from the second sieve bed that exceeds a pressure within a product tank in fluid communication with the second sieve bed to flow from the second sieve bed to the product tank; and
in response to completion of the third cycle portion, disconnecting fluid communication between the vacuum device and the first sieve bed, and substantially simultaneously opening a fluid flow path between the first sieve bed and the second sieve bed to perform a fourth cycle portion until the first sieve bed and the second sieve bed are at about equal pressure.

3. The method of claim 2 comprising:
completing at least one of the first cycle portion, the second cycle portion, the third cycle portion and the fourth cycle portion after a respective fixed time period.

4. The method of claim 2, comprising:
completing at least one of the first cycle portion, the second cycle portion, the third cycle portion and the fourth cycle portion after pressure in the product tank reaches a predetermined threshold.

5. The method of claim 2, comprising:
sequentially repeating the first cycle portion, the second cycle portion, the third cycle portion and the fourth cycle portion.

6. The method of claim 2, wherein each of the first sieve bed and second sieve bed are arranged such that a gas mixture passes through a porous material contained therein as the gas mixture passes from an inlet port of a respective sieve bed to an outlet port of the respective sieve bed, the porous material selectively absorbing at least one component of the gas mixture at a first higher pressure and desorbing the at least one component at a second lower pressure.

7. A method for providing a bolus output of oxygen to a medical patient comprising:
providing a vacuum pressure swing absorption (VPSA) oxygen concentrator apparatus including a product tank and a conserver reservoir;
providing multiple valves operable in a VPSA cycle, the multiple valves including an output valve;
providing an orifice between the product tank and the conserver reservoir to create a smoothed pressure curve in the product tank and reduce pressure variation in the conserver reservoir, the output valve pneumatically connected between the conserver reservoir and the medical patient interface;
determining pressure between the output valve and the medical patient interface; and
controlling timing of the output valve as a function of the pressure between the output valve and the medical patient interface to create a consistent bolus volume.

8. The method of claim 7, wherein the cycling of the output valve is performed in accordance with a lookup table of valve actuation times.

9. The method of claim 8, wherein the valve actuation times are provided in the table as a function of breath rate and a device setting.

10. A system for improved delivery of oxygen to a medical patient comprising:
a first sieve bed including a first pressure vessel having a first inlet port and a first outlet port, said first outlet port connected to a 2-way equalization valve;
a second sieve bed including a second pressure vessel having a second inlet port and a second outlet port, said second outlet port connected to the 2-way equalization valve;
the 2-way equalization valve having a first state providing a flow path between the first outlet port and the second outlet port, and having a second state closing the flow path between the first outlet port and the second outlet port;
a 4-way selector valve connected to the first inlet port, the second inlet port, a vacuum path and a compressed air path;
the 4-way selector valve having a first state providing a flow path between the compressed air path and the first inlet port and providing a flow path between the vacuum path and the second inlet port;
the 4-way selector valve having a second state providing a flow path between the compressed air path and the second inlet port and providing a flow path between the vacuum path and the first inlet port;
a 3-way vent valve connected in the vacuum path between a vacuum source and the 4 way selector valve and having a port open to atmospheric pressure;
the 3-way vent valve having a first state providing a flow path between the vacuum path and the vacuum source and having a second state providing a flow path between the vacuum source and the port open to atmospheric pressure while closing said vacuum path; and
a product tank having at least one product tank input port connected to the first outlet port through a first check valve and connected to the second outlet port through a second check valve, the first check valve arranged to allow flow from the first sieve bed to the product tank when pressure in the first sieve bed exceeds pressure in the product tank, the second check valve arranged to allow flow from the second sieve bed to the product tank when pressure in the second sieve bed exceeds pressure in the product tank.

11. The system of claim 10, comprising:
a conserver reservoir connected to the product tank via an orifice;
a 2-way conserver valve connected between the conserver reservoir and a patient interface;
the 2-way conserver valve having a first state providing a low path between the conserver reservoir and the patient interface; and having a second state closing the flow path between the conserver reservoir and the patient interface.

12. The system of claim 11, comprising:
at least one pressure transducer connected to the patient interface and arranged to measure pressure in the patient interface.

13. The system of claim 11, comprising at least one muffler in the compressed air path.

14. The system of claim 11, comprising at least one muffler in the vacuum path.

15. The system of claim 11, comprising:
a compressor and a filter, the compressor connected to the compressed air path and providing compressed air to the compressed air path via the filter.

16. The system of claim 10, comprising:
circuitry arranged for repeatedly switching from a first cycle portion to a second cycle portion to a third cycle portion to a fourth cycle portion;
wherein in the first cycle portion said switching sets the 2-way equalization valve to its first state, the 4-way selector valve to its first state and the 3-way vent valve to its first state;
wherein in the second cycle portion said switching sets the 2-way equalization valve to its second state, the 4-way selector valve to its first state and the 3-way vent valve to its second state;
wherein in the third cycle portion said switching sets the 2-way equalization valve to its first state, the 4-way selector valve to its second state and the 3-way vent valve to its first state; and
wherein in the fourth cycle portion said switching sets the 2-way equalization valve to its second state, the 4-way selector valve to its second state and the 3-way vent valve to its second state.

17. The system of claim 16 constructed as a self-contained portable apparatus.

18. The system of claim 16 wherein:
said circuitry is configured for completing at least one of the first cycle portion, the second cycle portion, the third cycle portion and the fourth cycle portion after a respective fixed time period.

19. The system of claim 16, comprising:
said circuitry configured for completing at least one the first cycle portion, the second cycle portion, the third cycle portion and the fourth cycle portion after pressure in the product tank reaches a predetermined threshold.

20. The system of claim 10, comprising:
circuitry arranged to control the respective states of the 2-way equalization valve, the 4-way selector valve and the 3-way vent valve to repeatedly switch from a first cycle portion to a second cycle portion to a third cycle portion to a fourth cycle portion;
the cycle portions including pressurizing the first sieve bed with air while substantially simultaneously evacuating the second sieve bed by applying the vacuum device to the second sieve bed for a first cycle portion;
during said first cycle portion, allowing gas from said first sieve bed that exceeds a pressure within the product tank in fluid communication with the first sieve bed to flow from the first sieve bed to the product tank;
in response to completion of the first cycle portion, disconnecting fluid communication between the vacuum device and the second sieve bed, and substantially simultaneously opening a fluid flow path between the first sieve bed and the second sieve bed to perform a second cycle portion until the first sieve bed and the second sieve bed are at about equal pressure;
in response to completion of the second cycle portion, pressurizing the second sieve bed with air while substantially simultaneously evacuating the first sieve bed by applying the vacuum device to the second sieve bed for a third cycle portion;
during the third cycle portion, allowing gas from the second sieve bed that exceeds a pressure within a product tank in fluid communication with the second sieve bed to flow from the second sieve bed to the product tank; and
in response to completion of the third cycle portion, disconnecting fluid communication between the vacuum device and the first sieve bed, and substantially simultaneously opening a fluid flow path between the first sieve bed and the second sieve bed to perform a fourth cycle portion until the first sieve bed and the second sieve bed are at about equal pressure.

21. The system of claim 20 constructed as a self-contained portable apparatus.

22. The system of claim 20 wherein said apparatus has an equalization point associated therewith, and wherein the apparatus includes components tuned so that the first sieve bed and the second sieve bed each have about equal pressure of about zero pounds per square inch at the equalization point.

23. The system of claim 11, wherein the cycling of the 2-way conserver valve is performed in accordance with a lookup table of valve actuation times.

24. The system of claim 23, wherein the valve actuation times are provide in the table as a function of breath rate and a device setting.

25. A vacuum pressure swing absorption (VPSA) type oxygen concentrator, comprising:
a first VPSA sieve bed connected to a product tank for supplying concentrated oxygen to the product tank, the first VPSA sieve bed including an input port configured to be closed or to receive either pressurized air or vacuum in response to switching of at least one valve;
a second VPSA sieve bed connected to the product tank for supplying concentrated oxygen to the product tank, the second VPSA sieve bed including an input port configured to be closed or to receive either pressurized air or vacuum pressure in response to switching of the valve;
wherein the first VPSA sieve bed is in switchable fluid communication with the second VPSA sieve bed via the valve;
control circuitry in communication with the valve, the control circuitry configured to switch the valve to open the switchable fluid communication between the first VPSA sieve bed and the second VPSA sieve bed and to allow pressure to equalize between the first VPSA sieve bed and the second VPSA sieve bed, the control circuitry configured to switch the valve between pressurizing the first VPSA sieve bed and pressurizing the second VPSA sieve bed substantially at the point when pressure in the first VPSA sieve bed equals pressure in the second VPSA sieve bed.

26. The oxygen concentrator of claim 25, comprising:
a control knob in communication with the control circuitry, wherein the control circuitry is configured to time switching of the at least one valve in response to the control knob setting.

27. The oxygen concentrator of claim 26, wherein the control circuitry is configured to control an amount of the pressurized air and an amount of vacuum pressure applied to the first VPSA sieve bed and the second VPSA sieve bed in response to the control knob setting.

28. oxygen concentrator of claim 25, wherein the control circuitry is configured to switching of the at least one valve in response to pressure in the product tank.

29. A simplified portable oxygen concentrator comprising:
a first VPSA sieve bed in communication with a product tank;
a second VPSA sieve bed in communication with the product tank;
at least one lightweight manifold attached to the first VPSA sieve bed and the second VPSA sieve bed, the manifold including flow paths between said first VPSA sieve bed, the second VPSA sieve bed and said product tank; and
at least one valve mounted on the manifold for controlling configuration of the flow paths;
wherein the product tank is adapted for fitting within a volume delimited by the first VPSA sieve bed, the second VPSA sieve bed, the manifold and the valve.

30. The simplified portable oxygen concentrator of claim 29, wherein the product tank comprises an irregular blow molded vessel.

31. The simplified portable oxygen concentrator of claim 30, wherein the product tank includes ultrasonically welded portions forming an improved sealing interface between the product tank and at least one of the flow paths.
